(19) United States
(12) Patent Application Publication
(10) Pub. No.: US 2005/0203490 A1
(43) Pub. Date: Sep. 15, 2005

(54) Apparatus and method for targeting for surgical procedures

(75) Inventor: Robert E. Simonson, Boca Raton, FL (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/125,763

(22) Filed: May 10, 2005

Related U.S. Application Data
(62) Division of application No. 10/112,784, filed on Mar. 29, 2002.

(51) Int. Cl. A61B 1/32

(52) U.S. Cl. 606/1

(57) ABSTRACT

A targeting instrument that consists of an indicating surface that is a planar body that can be placed so as to lie on the skin of a patient or placed in a cavity in the patient where an operation is being performed by a surgeon, so as to align the operation site with the target being operated on by the surgeon. The targeting instrument can be dimensioned to match the size and shape of the cannula or the desired size of the surgical exposure and can include a stencil configuration to allow the surgeon to inscribe the target area on the skin of the patient. The targeting instrument may include a handle. The method of use is to insert the targeting instrument in the location where the target of the operation is and to hone in on the target with the use of an imaging device. In one method of use this will align the target, select the entry incision and allowing the surgeon to make an accurate and minimally sized incision. In other methods of use, the targeting instrument can be placed in proximity to the skin or thereon of the patient for locating anatomical targets, for localizing bony anatomy.

### TECHNICAL FIELD

This invention relates to apparatus that is used to target a surgical incision site and more particularly to a targeting instrument that is utilized with an imaging device for placing the incision site in line with the area being operated on and the method thereof.

### BACKGROUND OF THE INVENTION

As is well known in this technology, one of the more difficult tasks for a surgeon performing spinal surgery or for that matter any type of surgery within the body of a patient, is to precisely select the incision site so that the location of the pathology or anatomical location being addressed is within or in-line with the incision site. One of the areas that is of particular concern is that of accurately placing implants at the targeted area. To best understand and appreciate the problems and concerns of the surgeon is to consider the surgical procedure a surgeon follows in, for example, large reconstructive spinal fusion, where the surgeon approximates the area of incision by gently approximating the anatomical landmarks, as for example, the iliac crest. The surgeon may perform an immediate incision on the area of concern or may otherwise wait to make an incision in conjunction with or depending on the outcome of an x-ray. The incisions are usually large and typically cover several vertebral levels, consequently, large or gross dissection of the soft paraspinal tissues is necessarily substantial.

Typically, the surgeon follows one of two techniques. In the first technique, the surgeon feels for an anatomical landmark on the patient. As mentioned above, for example, in a spinal surgery procedure, the surgeon would feel the iliac crest and then approximate the targeted anatomy and immediately begin the incision. At this point, the surgeon calls for an x-ray technician to come to the room and take a plain film x-ray image. Without exaggeration, the time involved before the technician arrives in the room, develops the x-ray film and returns to the room, more than ten (10) minutes have elapsed. While the technician is performing, the surgeon typically continues with the procedure by dissecting muscle and other soft tissue to expose the spine. Unless the x-ray shows the surgeon that he is on target, the surgeon will, of necessity, expand the incision in either direction, with the aim of reaching the intended target. It is readily apparent, that this procedure requires more dissection than would otherwise be required if precise targeting were available,

In another technique, the surgeon, after anesthetizing a patient, will insert a number of small needles, typically 2-4, in the back of the patient or alternatively, insert a wire rather than a needle or merely lay an object on the back of the patient. Like in the other technique, an x-ray will be taken and based on the x-ray, the surgeon will make an incision adjacent to the needle, wire or object, whatever the case may be, that is closest to the surgical target. While the first mentioned technique is the more widely used technique and the other technique is used with less regularity, consensus has it that the second technique is more accurate.

A significant effort has been made to assist the surgeon with inter-operative targeting and particularly directed to the accurate placement of implants. While there has been progress in this field, these apparatus are significantly sophisticated, typically utilizing a computer, infra-red camera, LED lights, and/or guide frames, etc., resulting in very expensive, complex hardware and requiring a significant amount of time to use. Moreover, the operators of this type of equipment needs to be versed in the use of this mechanism and often requires a number of courses before one could be considered an expert for operating this machinery.

As noted above, when the incisions are so large notwithstanding intuitive targeting or targeting by sophisticated mechanism where the area exposed by dissection is often many inches in length, the need for precision at this stage of the procedure is not very important. Hence, it is obviously apparent that where it is desirable to minimize the dissection of soft paraspinal tissue or the disruption of any other soft tissue, such procedures noted above are inadequate. Thus, many minimal invasive operations such as, microdiscectomies, laminectomies, foraminotomies, facetectomies, minimally invasive fusions, would require precision targeting in order to keep the disruption of soft tissue and the incision wound to a minimum.

I have found that I can precisely target the area being operated on with the targeting mechanism of this invention. Hence, with this invention, a more accurate method is utilized in providing targeting assistance prior to making the surgical incision. While experimental tests have shown that this invention is efficacious in a spinal procedure, as one skilled in this art will appreciate, it will also be efficacious in other areas of the body where targeting is of importance to the operating procedure.

In accordance with this invention, a generally planar circular or cross-hair or other identifying structure attached to a long handle, in one embodiment or without a handle in another embodiment, is placed over the skin of the patient in proximity to where the target is located and underneath an imaging device for the purpose of determining the anatomy below the skin level. The device is made so that a technician or surgeon can hold the instrument in the position noted immediately above and move it until it is in line with the target. The surgeon can then mark the skin with the use of this mechanism so that the incision will coincide with the markings and will be in-line with the target. It is contemplated within the scope of this invention, that the inventive surgical device and the method for its use, in addition to that described above, can be used as follows (these items are listed without intending to limit the scope of this invention):
with a plain film x-ray machine;
for determining the underlying anatomical target;
for localizing a surgical entry point;
for use in an operative environment by being held in close proximity to the patient's skin (not necessarily requiring attachment to the patient) and used in conjunction with x-ray imaging;
for localizing bony anatomy by placing a specialized instrument in close approximation to a patient and utilizing an x-ray imaging device;
for targeting the spine by placing a hand-held targeting device between the patient's anatomical target and imaging device;
for targeting the spine for the purpose of choosing an entry point by placing a hand-held targeting device between the patient's anatomical target and an imaging device.

It is contemplated that this invention will be made in both a disposable and reusable model; that the material could be metal or plastic or a combination thereof; that it could be made to retain ink or other marking ingredient for providing a temporary stain on the patient prior to contact; it could include numerical markings on the instrument which can be employed to allow for measurements to be taken; one embodiment of this invention will have a space or outline such as a stencil or template to permit the drawing of a line or lines on the skin of a patient with a marking pen; and the long shaft of the instrument could be placed laterally beside the patient to determine surgical level. The instrument of this invention, also, has utility in a procedure where the incision precedes the targeting.

### SUMMARY OF THE INVENTION

An object of this invention is to provide an instrument used to target an area intended to be operated on beneath the skin of a patient.

An object of this invention is to provide a hand-held instrument that is usable with a plain film x-ray machine or imaging machine for aligning the point of entry into the patient that aligns with the target being operated on by the surgeon. In another embodiment, the instrument may be a uniquely configured instrument that is placed on the skin of the patient and the handle being an option.

A feature of this invention is a planar indicating portion extending from a handle in one embodiment and without a handle on another embodiment. The indicating portion could be a template with a guiding portion for allowing the passage of a marking instrument to identify the area of the incision on the skin of the patient. In a circular configuration the diameter of the circle portion will coincide with the diameter of a cannula intended to be used in the surgical procedure. Another configuration can be a cross hair with or without a stencil portion for marking the target area with a pen or other marker. The instrument could be made from metal, plastic or a combination of both, could be either disposable or re-usable and is characterized as being relatively inexpensive to manufacture, easy to manufacture and use and has the propensity of requiring a minimum amount of time than is required for heretofore known techniques and targeting equipment.

The foregoing and other features of the present invention will become more apparent from the following description and accompanying drawings.

### BRIEF DESCRIPTION OF THE DRAWINGS

FIG. 1 is a plan view showing the details of one embodiment of this invention;

FIG. 2 in a plan view showing the details of another embodiment of this invention;

FIG. 3 is a plan view showing the details of another embodiment of this invention;

FIG. 4 is a plan view showing the details of another embodiment of this invention;

FIG. 5 is a plan view showing the details of another embodiment of this invention; and

FIG. 6 is an artist's rendition of the 3.sup.rd and 4.sup.th lumbar vertebrae posterior view and the invention as would be viewed from an imaging machine.

These figures merely serve to further clarify and illustrate the present invention and are not intended to limit the scope thereof.

### DETAILED DESCRIPTION OF THE INVENTION

This invention is shown in its preferred embodiment with a given number of different configurations, and as one skilled in this art will appreciate, the particular configuration is one of choice and this would typically be made by the surgeon. It will be understood that the intent of this disclosure is to show how this invention would be employed as a targeting device and the particular configuration of the device or instrument should not alter the scope of this invention. In one embodiment, the configuration of the instrument is a circle whose diameter is equal to the diameter of the cannula that will be used in the surgical procedure. Obviously, the configuration could be other than a circle, as for example an ovoid, or any polygonal configuration, etc. without departing from the scope of this invention. Further, it will be appreciated that in certain operating procedures, the physician utilizes a skin drape to place various markings on the patient or otherwise marks directly on the skin, in accordance with this invention, either procedure is covered by the scope of this invention.

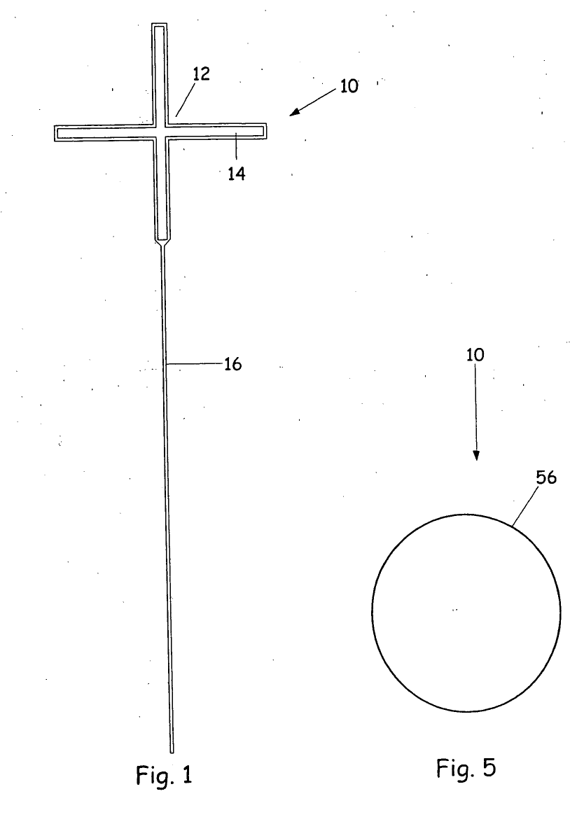

Reference is now made to all of the Figs. which best describes this invention where the targeting instrument is generally referred to as reference numeral 10 in all of the different embodiments. In FIG. 1 the targeting instrument 10 includes an indicating portion 12 and a handle 16. The indicating portion 12 is generally planar and is generally shaped similar to a cross and includes an open inner passageway 14 designed similar to a stencil so that a marker or pen can be used to mark the area that has been targeted. A handle 16 may be attached to one end of the indicating portion 12, although, the use of a handle is an option. The handle 16 could be in-line with the indicating portion 12 or off-set. In the preferred embodiment, the indicating portion is configured with a small wire that is bent similar to the well known paper clip into the desired configuration or alternatively, metal or plastic material can be stamped out to form the desired configuration.

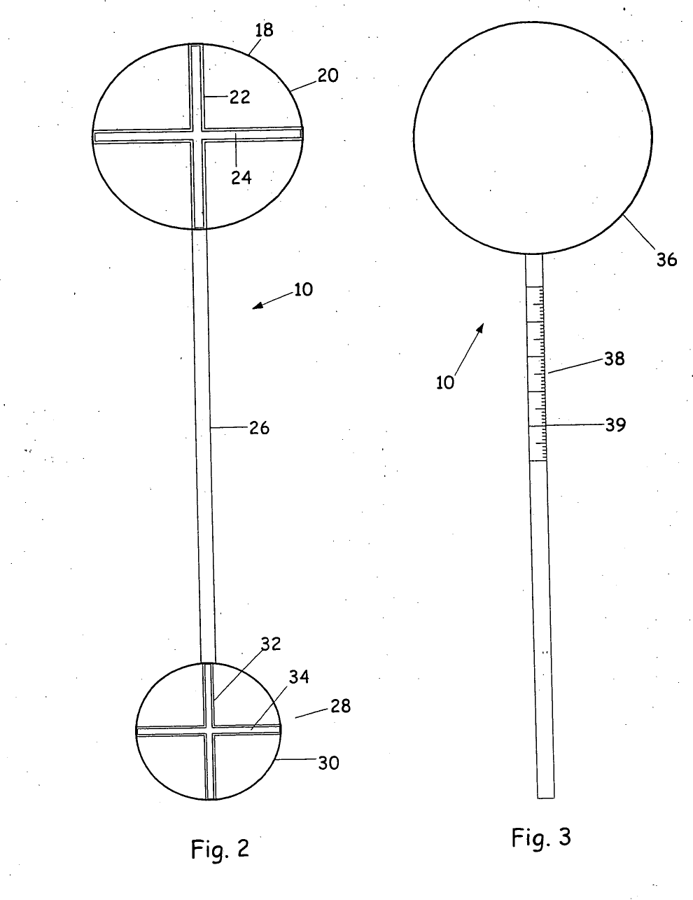

FIG. 2 exemplifies another embodiment where the instrument includes different sizes. At the upper end 18 of handle 26 the indicating mechanism includes a circular ring 20 and a cross-hair portion 22. The cross-hair configuration includes a open passageway or stencil 24 for accommodating a marking instrument. The lower end 28 of handle 26 carries an identical structure but of a different size. Hence, it includes the circular ring 30, the cross-hair portion 32 and the open passageway or stencil 34. It will be appreciated that the diameters of rings 20 and 30 are selected to mimic the diameter of the cannula or the size and dimensions of the surgical exposure that will be used in the surgical procedure.

FIG. 3 exemplifies still another embodiment where the instrument includes ring 36 attached to handle 38. Again, the diameter of the ring 36 is designed to match the size of the cannula or the size and dimensions of the surgical exposure that will be used in the surgical procedure. Each of the handles, if desired, could include numerical indicia 39 for indicating distances.

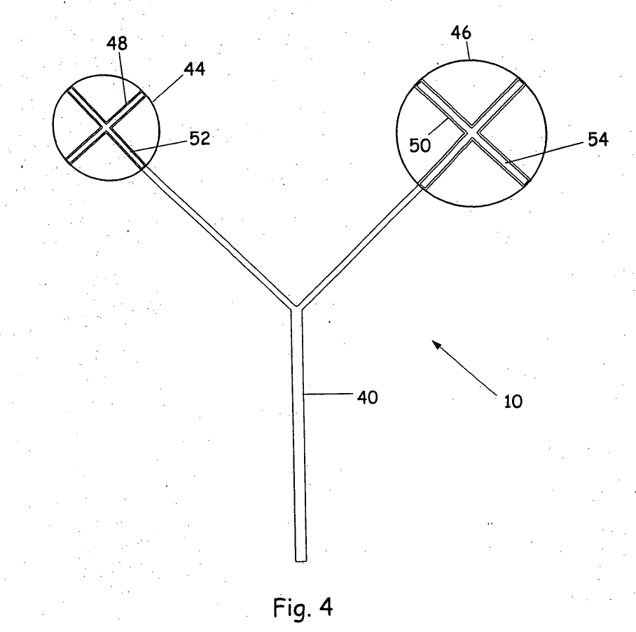

FIG. 4 is another embodiment where the handle 40 is configured in a “Y” shape and carries indicating mechanism at the end of each arm of the Y. Hence, similar to FIG. 2 the rings 44 and 46 are sized to match the cannula or the size and dimensions of the surgical exposure being used in the surgical procedure and the cross-hair configurations 48 and 50 include the open passageways 52 and 54 to accommodate a marking pen.

FIG. 5 is still another embodiment of this invention and is symbolic of all the other embodiments that could be made without handles. In this embodiment the size of the circular ring 56 is designed to match the size of the cannula or the size and dimensions of the surgical exposure being used in the medical procedure.

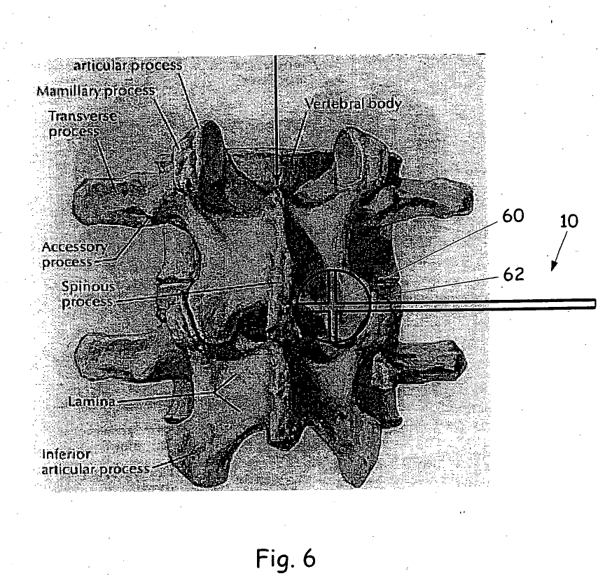

In operation of this invention, reference should be made to FIG. 6 which is illustrative of a plain film x-ray after the targeting instrument 10 is placed on the patient in proximity to the target of the surgery where the operation will occur. In this example, the identification of the target and targeting device is shown by what would otherwise be a plain film x-ray of the 3^{rd} and 4^{th} lumbar vertebrae. Obviously, in other imaging devices where the picture is shown on a screen or monitor the surgeon would position the targeting instrument to align with the target and proceed with the operation. It is apparent from the foregoing that the cross hair portion 60 of the indicating portion 62 of the targeting instrument 10 will align with the target, thus giving a longitudinal and latitudinal position. Not only will this target the point of surgery, it will also be a guide for the size of the incision. The surgeon, of course, can mark the skin of the patient with the use of the stencil portion of the targeting instrument. In practice, if the targeting instrument is not in the proper location, the instrument will be moved until it is in line with the target. Hence, it is apparent that the incision that will be made will be directly above or in coincidence with the target. This procedure eliminates the necessity of enlarging the incision if the incision was not in line with the target or otherwise inaccurate. In practice before this invention, the inaccurate targeting of the initial surgical incision could double the necessary size of the incision. This of course, disrupts more healthy tissue which can be the cause of morbidity, longer hospital stays, more pain medication, longer recovery periods and other issues related to a more invasive technique. By taking lateral views of the targeting instrument, the chances of performing the surgical procedure on the wrong vertebral level is reduced, if not eliminated. In actual practice I have found that when this invention has been clinically tested when this invention together with taking anterior/posterior and/or lateral views of the targeting instrument when in the vicinity of the surgical target performing the surgical procedure on the wrong target has been eliminated, and hence, the chances of performing the surgical procedure on the wrong target will be reduced, if not eliminated. The occurrence of operating the surgical procedure on the wrong vertebral level is well known and documented in the surgical journals.

The following is an example of the procedure that would be followed by a surgeon when utilizing this invention.
1) The surgeon decides how much space will be required as a working area.
2) The operator or surgeon will hold the handle of the targeting instrument and place the indicating portion of the targeting instrument over the target area and will hold it vertically beside the patient and a lateral x-ray will be taken. This will clearly identify the vertebral level.
3) The indicating portion of the targeting instrument will be held across the patient's back while an x-ray is being taken.
4) the circular portion or ring configuration and the cross-hair portion of the targeting instrument will clearly appear in the x-ray image. When it is over the target, the surgeon continues with the next step in the surgical procedure. Obviously, if it isn't over the target, the surgeon will reposition it, take another image and continue this procedure until it is.
5) In this example, the passageway or stencil allows the surgeon to take a common skin pen and mark an “X” or cross over the skin to clearly mark the surgical site.
6) Inasmuch as the surgeon will know the exact center of the surgical target, he can make an accurate incision size.

It is apparent from the foregoing that many procedures can take advantage of this targeting instrument.
a) For example, this targeting instrument can be held between the patient and an imaging device for the purpose of determining anatomy below the skin level
b) This targeting instrument can be held in the hand of the surgeon or a technician and placed between the patient's skin and an imaging device.
c) This targeting instrument can be held between a patient's skin and an x-ray device for the purpose of determining the location of underlying anatomical targets.
d) This targeting instrument can be held between the patient's skin and x-ray device for the purpose of localizing a surgical entry point.
e) This targeting instrument can be held in close proximity to a patient's skin in an operative environment, not necessarily requiring attachment to the patient, and used as a targeting device when used in conjunction with x-ray imaging.
f) This targeting instrument can be used for localizing bony anatomy by placing this unique invention in close approximation to a patient and utilizing an x-ray imaging device.
g) This targeting instrument could be used on a patient beneath the skin surface for targeting the spine by placing a hand held targeting device between the patient's anatomical target and an imaging device.

What has been shown by this invention is a simple, yet useful instrument that can be used in operating and identifying medical procedures where targeting is of importance. While only a few species of targeting instruments were depicted in this patent application, the number and types of targeting instruments are many. As has been emphasized in the descriptive material of this patent application, the configurations of these targeting instruments can be predicated on the size of the operation, the type of operation, the location of the malady, the preference of the surgeon. The instrument could include a stencil or not, a handle or not, or include the ink or the provision to accommodate a marking pen. It could be configured to match the size and shape of the retractor or cannula being used in the operation, and a single handle could incorporate a plurality of indicating portions with different sizes and shapes.

Although this invention has been shown and described with respect to detailed embodiments thereof, it will be appreciated and understood by those skilled in the art that various changes in form and detail thereof may be made without departing from the spirit and scope of the claimed invention.

1-18. (canceled)

19. A method of targeting an anatomical location for performing an operation on a patient where the incision is at a minimal size including the steps of:
i) providing a targeting instrument that is configured to lie on the skin of a patient and to locate the incision to be made;
ii) placing the targeting instrument on the skin of the patient in the vicinity of the location of the anticipated anatomical target; and
iii) providing an imaging device to view the anatomical target and the targeting instrument and positioning the targeting instrument to align with the anatomical target.

20. The method of targeting an anatomical target location as claimed in claim 19 including the step of providing a stencil portion on the targeting instrument, and inserting a marking on the skin of the patient with the use of the stencil to indicate the point of incision.

21. The method of targeting an anatomical target location as claimed in claim 19 including the step of providing a targeting instrument that is sized and shaped to match the cannula intended to being used in the operation.

22. The method of targeting an anatomical location as claimed in claim 19 including the step of providing a targeting instrument that is sized and shaped to match the desired size and/or dimension of the surgical exposure.

23. The method of targeting an anatomical location as claimed in claim 19 including the step of providing a targeting instrument with a handle for the user to position the targeting instrument to align with the anatomical location.

24. The method of targeting an anatomical location as claimed in claim 19 including the step of imaging the lateral position of the targeting instrument.

25-34. (canceled)

35. A method for targeting a surgical site, comprising:
positioning a targeting instrument having a size that corresponds to a size of a cannula intended to be used in a surgical procedure above the skin of a patient in proximity to a target where an incision is to be made;
determining a location where an incision is to be formed by aligning the targeting instrument with a target surgical site using an imaging instrument; and
forming an incision at the determined location.

36. The method of claim 35, further comprising marking the skin at the location where an incision is to be formed prior to forming an incision at the determined location.

37. The method of claim 36, wherein the targeting instrument includes an open passageway and the marking is formed through the open passageway.

38. The method of claim 35, wherein the target is a vertebra.

39. The method of claim 35, wherein aligning the targeting instrument with a target surgical site using an imaging device comprises viewing the targeting instrument and the target on an x-ray image, and moving the targeting instrument until it is aligned with the target.

40. A method for targeting a surgical site, comprising:
positioning an incision template of a targeting instrument above a patient's skin;
viewing the incision template and a target surgical site using an imaging instrument;
aligning the incision template with the target surgical site; and
forming a marking on the patient's skin through the incision template.

41. The method of claim 40, wherein the marking is formed through a cross-shaped opening formed in the incision template.

42. The method of claim 40, wherein the incision template is positioned above the patient's skin by holding a handle extending from the incision template.

43. The method of claim 40, wherein the incision template has a size that corresponds to a size of a cannula to be inserted through an incision formed in the patient's skin.
